(19) United States
(12) Patent Application Publication
(10) Pub. No.: US 2026/0359498 A1
(43) Pub. Date: Nov. 12, 2026

(54) METHODS AND SYSTEMS FOR AGROCHEMICAL MANAGEMENT

(71) Applicant: SYNGENTA CROP PROTECTION AG, Basel (CH)

(72) Inventors: Raymond Joseph WUERFFEL, Greensboro, NC (US); Alissa KRISS, Basel (CH); Taylor GLENN, Greensboro, NC (US); Rob LIND, Bracknell, Berkshire (GB)

(73) Assignee: SYNGENTA CROP PROTECTION AG, Basel (CH)

(21) Appl. No.: 19/134,544

(22) PCT Filed: Nov. 30, 2023
(86) PCT No.: PCT/US2023/081777

§ 371 Date: May 30, 2025

Related U.S. Application Data
(60) Provisional application No. 63/385,463, filed on Nov. 30, 2022.

(51) Int. Cl. G06F 30/13; A01N 35/06; A01N 37/22; A01N 43/40; A01N 43/70; A01N 57/20; A01P 13/00; G01N 33/00; G06Q 50/02

(52) U.S. Cl. CPC G06F 30/13; A01N 35/06; A01N 37/22; A01N 43/40; A01N 43/70; A01N 57/20; A01P 13/00; G01N 33/0098; G06Q 50/02

(57) ABSTRACT

The present disclosure relates to methods and systems for managing agrochemical application.

### TECHNICAL FIELD

The present invention relates to methods and systems for monitoring field conditions. In particular, the methods and systems relate to monitoring weed growth in fields.

### BACKGROUND

To meet demand, modern agriculture relies on agrochemical compositions to protect and enhance crop growth. However, determining the amount of agrochemical needed to support crop growth is often difficult due to the variability in the large number of parameters which determine how much agrochemical is needed, e.g.: pest pressure, resistance, previous agrochemical applications, growth rates of crops, weather, etc.

Moreover, it is not beneficial to buy too much or to little agrochemical. Agrochemical compositions do not have an indefinite shelf-life, and unused agrochemical compositions either need to be disposed of or stored, at the risk of failure, until the next growing season. Excess agrochemical cannot be applied if it exceeds regulatorily approved amounts. If too little agrochemical is acquired, the window for agrochemical application may pass by the time more agrochemical is received and applying too little agrochemical can lead to resistance or loss in profits based on untreated areas.

Accordingly, the inventors desire methods and systems for managing agrochemical application.

### SUMMARY

Embodiments includes methods and systems which involve scanning a field with a drone having at least one sensor, and identifying a presence of wanted or unwanted plant growth in the field.

### BRIEF DESCRIPTION OF DRAWINGS

FIG. 1A shows stitched images of a field taken from a drone.

FIG. 1B shows an overlay of weeds and crops on the stitched images of FIG. 1A.

FIG. 2 shows identification of weeds between pre-emergent applications of Bicep II Magnum® herbicide versus Acuron® herbicide.

FIG. 3 shows identification of weeds during V1, V3, and V5 growing stages of corn.

FIG. 4 shows the simulated % area needed for spraying over various locations.

FIG. 5 shows identification of weeds at various spray resolutions.

FIG. 6 shows the impact of spray resolution on areas sprayed in simulated V5 growth stage of corn.

### DETAILED DESCRIPTION

Before certain embodiments are described in greater detail, it is to be understood that this disclosure is not limited to certain embodiments described, as such may, of course, vary. It is also to be understood that the terminology used herein is for the purpose of describing certain embodiments only, and is not intended to be limiting, since the scope of the present disclosure will be limited only by the appended claims.

Described herein are several definitions. Such definitions are meant to encompass grammatical equivalents.

The use of “or” means “and/or” unless stated otherwise. Furthermore, the use of the terms “comprising,” “having,” “including,” as well as other forms, such as “includes” and “included,” are intended to be inclusive and mean that there may be additional elements other than the listed elements.

As used herein, the term “about” when referring to a measurable value such as an amount, a temporal duration, and the like, is meant to encompass variations. Such variations, however, are dependent on the specific component referred to and the context as understood by a person of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, representative illustrative methods, and materials are now described.

Each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Embodiments of the disclosure can be practiced without any component not specifically mentioned in this disclosure.

Embodiments described herein may comprise, consist essentially of, or consist of the elements therein.

Unless otherwise stated percentages are given as percentages by total weight and all embodiments and preferred features may be combined in any combination.

Optical spot spraying technology is becoming more common in the marketplace. Understanding how this technology may perform in an integrated weed management systems is complex, and access to research equipment is currently a challenge. Furthermore, it was unclear that high resolution drone imagery could be used to simulate postemergence applications using an optical spot sprayer following preemergence applications.

Following the preemergence application, drone flights were performed at the V1, V3, and V5 corn growth stages. Vegetative or V stages are described in more detail below. Images from each flight were then converted and analysed to simulate postemergence optical spot spray applications. The simulated post sprays indicated greater weed control from Acuron® herbicide compared to Bicep II Magnum® herbicide. The resulting simulated postemergence spray volume savings aligned well with previous field results. The results from these trials indicate that the use of high-quality drone imagery can be used to simulate postemergence optical spot spraying applications. Furthermore, these data indicate that early season weed management will remain critical to weed management in a future where optical spot sprayers are used.

The V stages of corn occur at specific intervals of time and mark the developmental progress of the corn plant before it transitions to the reproductive stages. The V stages begin with V1, which typically occurs within 2 to 3 weeks after planting when the first leaf emerges and unfolds. As the plant progresses through V2 and V3, additional leaves develop, and the stem and root system continue to grow. By V4 and V5, which occur around 3 to 4 weeks after planting, the plant exhibits more leaves and an increased height. During V6 and V7, approximately 4 to 5 weeks after planting, the corn plant experiences rapid growth, with more leaves, a well-developed stalk, and an expanding root system. The V8 stage, around 5 to 6 weeks after emergence, is characterized by the completion of leaf development, and the plant enters the final vegetative stage, V9. At this stage, which occurs around 7 to 9 weeks after planting, the corn plant focuses on accumulating energy reserves and preparing for the reproductive phase. Proper management practices during the V stages, including timely nutrition, adequate water supply, and effective weed control, are crucial to ensure optimal growth and maximize the corn plant's potential for a successful reproductive stage and yield.

The present disclosure is not limited to corn. Within the scope of present disclosure, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The term “agrochemical” or equivalent terms include compounds or ingredients registered as being biologically active against an agricultural pest. In general, agrochemical active ingredients include compounds listed in: The Pesticide Manual, 12th edition, 2001, British Crop Protection Council. Agrochemicals include, but are not limited to herbicides, fungicides, other insecticides, bactericides, insect growth regulators, plant growth regulators, nematicides, molluscicides or mixtures of several of these preparations. In preferred embodiments, the agrochemical is an herbicide.

Examples of herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1 (2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl) pyrazol-3-yl]imidazolidin-2-one, (4R) 1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl 2-[[3-[[3-chloro-5-fluoro-6-[3-methyl-2,6-dioxo-4-(trifluoromethyl)pyrimidin-1-yl]-2-pyridyl]oxy]acetate and 6-chloro-4-(2,7-dimethyl-1-naphthyl)-5-hydroxy-2-methyl-pyridazin-3-one.

The term “agrochemically effective” or equivalent terms generally refer to approved rates of application of an agrochemical. An agrochemically effective amount is generally determined by the specific agrochemical and target thereof being used.

The rates of application of agrochemicals may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop.

The application is generally made by spraying a composition, typically by tractor mounted sprayer for large areas, but other methods such as dusting (for powders), drip or drench can also be used.

Agrochemical compositions of the disclosure can have a sole active ingredient or can be admixed with one or more additional active ingredients. An additional active ingredient may, in some cases, result in unexpected synergistic activities.

The compositions of the disclosure may be employed in any conventional form, for example in the form of a twin pack, an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants. In general, such compositions are concentrates and diluted prior to use, however, composition can be provided in ready-to-use form (i.e., no dilution required).

In general, the concentrate compositions include from 0.01 to 90% by weight of active agent, from 0 to 20% agriculturally acceptable surfactant and 10 to 99.99% solid or liquid formulation inerts and adjuvant(s). Concentrated forms of compositions generally contain in between about 2 and 80%, preferably between about 5 and 70% by weight of active agent.

Application forms of compositions can, for example, contain from 0.001 to 20% by weight, or preferably from 0.002 to 5% by weight of active agent. High precision application techniques may allow for higher concentrations of agrochemicals.

The term “safener” as used herein means a chemical that when used in combination with a herbicide reduces the undesirable effects of the herbicide on non-target organisms, for example, a safener protects crops from injury by herbicides but does not prevent the herbicide from killing the weeds. Compositions of the disclosure can include a safener. The following safeners are especially preferred: benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

In preferred, herbicide applications, the compositions of the disclosure can typically be used to control a wide variety of monocotyledonous and dicotyledonous weed species in the crop. The compositions of the present invention provide particular good control of Alopecurus sp. (e.g Alopecurus myosuroides (ALOMY)), Avena sp. Digitaria sp. (e.g Digitaria sanguinalis (DIGSA), Digitaria insularis (TRCIN)), Echinochloa sp. (e.g Echinochloa crus-galli (ECHCG)), Eleusine sp. (e.g Eleusine indica (ELEIN)), Lolium sp., Setaria sp. (e.g Setaria faberi (SETFA)) and Sorghum sp. (e.g Sorghum halepense (SORHA). In all aspects of the disclosure, in any particular embodiment, the weeds, e.g. to be controlled and/or growth-inhibited, may be monocotyledonous or dicotyledonous weeds, which are tolerant or resistant to one or more herbicides for example, HPPD inhibitor herbicides such as mesotrione, PSII inhibitor herbicides such as atrazine or EPSPS inhibitors such as glyphosate.

Within the present disclosure are methods and systems for evaluation of the effectiveness of agrochemicals. The techniques described herein can be used to evaluate the effectiveness of both pre-emergent agrochemicals and post-emergent agrochemicals, as well as the techniques themselves for pre/post-emergent application. As used herein, the term “pre-emergent,” and the like, refers to the period of time before the emergence of the crop, and the term “post-emergent,” and the like, refers to the time period after the emergence of the crop. Preferred methods and systems disclosed herein are post-emergent.

Embodiments of the disclosure additionally include methods and systems for metering agrochemical application. Metering of agrochemicals, as used herein, refers to predictively determining an amount of agrochemical needed for effective treatment of a field. While metering of agrochemicals may be relevant to broadcast application, for example, based on total disease pressure of a field or the presence of resistant pests, metering of agrochemicals is preferably associated with variable application techniques, for example, spot spraying.

Additional aspects and embodiments of the disclosure relate to methods and systems for assisting farmers in making field management decisions. Field management refers to management of a field prior, during, or after agrochemical application. Field management can include selecting an agrochemical for application and/or determining areas for agrochemical application.

Various agrochemical application techniques are known in the art. Preferred embodiments of the disclosure relate to spot spraying techniques used in post-emergence applications. Spot spraying is particularly useful when the agrochemical is an herbicide.

It is contemplated that herbicides can also be used for pre-harvest desiccation in crops, for example, but not limited to, potatoes, soybean, sunflowers and cotton. Pre-harvest desiccation is used to desiccate crop foliage without significant damage to the crop itself to aid harvesting.

Embodiments of the disclosure can include a step of analysing a field. The analysing can occur prior to agrochemical application, during agrochemical application, or after agrochemical application. Analysing a field can include measuring at least one of mineral/nutrient/fertilizer content, residual agrochemicals/contaminants, microbial populations, moisture content, and/or weeds. Such measurements can be obtained from direct analysis of the soil. In some embodiments, analysis can include measurement of ambient conditions, for example, temperature, humidity, brightness, etc.

Embodiments of the disclosure can include applying an agrochemical to a field. In some embodiments, this application can be pre-emergent. However, the application can also be post-emergent. In embodiments where the applying of the agrochemical to the field is post-emergent, there can preferably be a second post-emergent application of a second agrochemical. In preferred embodiments, the agrochemical used in the first post-emergent application is different from the agrochemical used in the second post-emergent application.

Aspects of the disclosure include scanning the field with a drone. In some embodiments, the scanning can be part of analysing the field. In general, the drone will include at least one sensor for measurements of the field. In specific embodiments, the at least one sensor is camera. The at least one sensor, however, can also include other means of characterizing the field. For example, sensors can include LIDAR or other sensing equipment which can be used to differentiate and/or characterize the contents of a field. Drones can be fully autonomous, partially autonomous, or remote controlled.

Drones, aerial and terrestrial, are generally known in the art. For example, drones for agricultural use are disclosed in U.S. Pat. Nos. 10,364,029; 11,144,059; 10,599,959; 9,382,003; and 9,563,852; US Patent Application Publications: 20220073205; 20170231213; 20160050840; 20160176542; 20170231213; 20180068164; and 20180068165. All of which are incorporated by reference in their entirety herein.

In certain embodiments, the sensor can be included in agricultural equipment. As used herein, agricultural equipment refers to equipment used in a field in relation to the production of crops. Drones, as described herein, are included as agricultural equipment.

Precision agriculture equipment is also generally known in the art. For example, U.S. Pat. Nos. 11,445,658; 10,645,866; 10,561,059; 9,980,429 US Patent Application Publications: 20160253595; 20180359909; 20210112698; 20210059109; 20210307242. All of which are incorporated by reference in their entirety herein.

Using measurements taken from the at least one sensor of the agricultural equipment, systems and methods of the disclosure use the information to identify the presence of wanted or unwanted plant growth in field. Unwanted plant growth includes not only weeds, per se, but also volunteer/escape crops.

The identification can take place either directly on the agricultural equip, or the scanned information can transferred to a computer for processing and analysis. By way of example, a system of the disclosure can include a drone for scanning a field, precision agricultural equipment for applying an agrochemical, and a computer for coordinating and processing information received from the drone and precision agricultural equipment. In some embodiments, the computer is part of a cloud system. Alternatively, all processing can be done onboard a single piece of agricultural equipment, and information and decision requests can be sent to a user interface.

A spot spray simulation can be generated from the identification of the wanted and unwanted plant growth. Spot spray simulations generally refer to a theoretical application of agrochemicals, in particular, using an optical spot spray technique. In some embodiments, the methods and systems of the disclosure can determine whether to broadcast/apply agrochemical to an entire field, for example, if disease pressure in the field is too high (above a threshold level).

The spot spray simulation can take into account a variety of different agrochemicals for application.

Spot spray simulation include non-spot application zones (NZs) and simulated spot applications zones (SAZs). NZs are areas where it is desirable not to apply agrochemical. SAZs are areas where it is desirable to apply agrochemicals. In general, SAZs correspond to unwanted plant growth, whereas NZs include wanted plant growth and/or areas with no growth. In some embodiments, SAZs can include wanted plant growth, for example, if a plant growth regulator is the agrochemical being applied.

For ease of processing, the spot spray simulation can be two-dimensional, an X-Y axis analysis. However, it is contemplated that the spot spray simulation can include at least three-dimensions, at least four-dimensions, or more. An example of a third dimension would be a Z-axis. A third dimensional Z-axis may be useful for adjusting the total amount of agrochemical to be applied depending on the size of wanted or unwanted plants. An example of a fourth dimension is time. Spot spray simulations can take into account the amount of time necessary to treat a field. Time can be relevant to compositions which are less stable. For less stable compositions, it may not be possible to treat an entire field prior to composition separation or failure. Time may also be relevant to systems where end users rent equipment. Estimations of equipment usage may allow owners of farm equipment to maximize their rental time, as well as save money for end users.

In specific embodiments, NZs and SAZs are configured based on a spray resolution. A spray resolution refers to the accuracy of the agrochemical application equipment, in particular, a sprayer. Spray systems generally include a reservoir which holds an agrochemical, a pressurization system or other methods for dispensing material (e.g., gravity), and a nozzle or exit to direct the agrochemical. NZs and SAZs, and SAZs in particular, can be divided into areas which are discretely based on the spray resolution. The spray resolution value includes at least one of 25×25 cm; 50×50 cm; 100×100 cm, or a resolution in the range of 5-100 cm×5-100 cm.

In some embodiments, NZs and SAZs can have irregular shapes. In such embodiments, NZs and SAZs trace the perimeter of wanted or unwanted plant growth. The use of irregular zones can be associated with a applications that have a higher degree of precision. In some embodiments, NZs and SAZs are initially traced as irregular shapes and converted in a polygon (square or rectangle) or circular shape for the spot spray simulation. The polygon or circular shape can be selected based on the spray from sprayer. In general, where irregular shapes are converted into a polygon or circular shape, the perimeter of the polygon or circular shape encompasses the perimeter of the irregular shape.

In specific embodiments, the spray from the nozzle is ring shaped. The use of a ring-shaped spray may be beneficial where it is desirable to spray around wanted plant growth. Alternatively, many agrochemical function systemically, and it is therefore advantageous to focus a majority of the agrochemical application on the leaves of wanted or unwanted plant growth.

A spot spray simulation can be used to assist a user in determining an appropriate nozzle type for spot spray application.

In some embodiments, the NZs are determined by subtracting SAZs from a theoretical broadcast/full coverage spray system, or vice versa. For example, the spot spray simulation can first generate a report for broadcast/full coverage spray of a field. The report can include information such as the total amount of agrochemical used, water, energy inputs (e.g., gas or electricity), time, etc. In such embodiments, the report treats agrochemical application as being evenly applied across the entire field. Alternatively, the broadcast/full coverage analysis can take into account field specific factors, e.g., wind and terrain, which may result in “hot spots” of application. A hot spot refers to an overapplication of agrochemical. After scanning the field with the drone, and identifying the wanted or unwanted plant growth, the areas identified with unwanted plant growth are marked in the report, and all other areas are subtracted leaving only SAZs. The spot spray simulation can be further processed in accordance with the systems and methods described herein.

Spot spray simulations can be provided to users as a report and/or as a data set for additional processing. In any case, the spot spray simulation can have a matrix and/or grid of wanted and unwanted plant growth. A matrix refers to a rectangular array or table of numbers, symbols, or expressions, arranged in rows and columns, which is used to represent a mathematical object. In contrast, a grid refers to a graphical representation: a diagram showing the relation between variable quantities, typically of two variables, each measured along one of a pair of axes at right angles. In general, the grid is cartesian and positions of the grid represent X-Y coordinates of a plot having plant growth. However, other systems can be used, e.g. polar coordinate systems. Polar coordinate systems can be advantageous in calculating efficient routes for agrochemical application.

Specific embodiments of the disclosure includes applying a first agrochemical to a first field. A crop can be plant before or after the applying of the first agrochemical to the first field. The applying of the first agrochemical to the first field can preferably be a pre-emergent application, however, in some embodiments, the application can be post-emergent.

The first field can be scanned both before and after the first agrochemical is applied. Scanning the field before agrochemical application allows a user to determine possible pest pressure. Scanning after the first agrochemical is applied can allow for both an assessment of pest pressure and effectiveness of the first agrochemical/application.

In general, the first field is planted at a time T and scanning occurs during at least one of 1-12 weeks after T, 2-4 weeks after T, 4-6 weeks after T, 6-8 weeks after T, or 9-10 weeks after T.

The scanning can be done by a first drone having at least one sensor. Based on the scanning, identification of wanted or unwanted plant growth in the first field is made. Based on the identification, a first spot spray simulation is made for a second agrochemical based on the presence of wanted or unwanted plant growth in the first field. The spot spray simulation can be used to evaluate the effectiveness of the first agrochemical, for example, by analyzing the amount of a second agrochemical needed. In preferred embodiments, at least one or both of the first agrochemical and the second agrochemical include an herbicide.

Embodiments of the disclosure can include applying a third agrochemical. In some embodiments, the third agrochemical is applied to a second field. The second field can be a portion of the first field or an entirely separate field. In some embodiments, the third agrochemical is different from the first agrochemical. For example, embodiments of the disclosure contemplate where the first agrochemical and the third agrochemical are both pre-emergent herbicides, and the second agrochemical is a post-emergent herbicide. In such embodiments, the first and third agrochemical can be directly compared to one another in separate fields, or the second field can stack the application of the first and third agrochemical to determine an amount of improvement in pest control. One criterion for evaluation of the effectiveness of the application of the first and/or third agrochemical is the amount of the second agrochemical needed.

The second field can be scanned with a second drone having at least one sensor. The second drone can be the same or different from the first drone.

In methods and systems of the disclosure which relate to generating estimates of an amount of the second agrochemical for application, such systems and methods, in addition to applying the second agrochemical, can measure the amount of the second agrochemical applied, and further report a difference between the volume applied and the volume estimate. The differences between volume applied and volume estimate can be used to improve the accuracy of the estimate.

Methods and systems of the disclosure can include compiling spot spray simulations. The spot spray simulations can be used to create a database. In specific embodiments, the database of spot spray simulations can be matched to a specific agrochemical. Other parameters can be used to designate spot spray simulations, e.g., related locations/terrains, weather, pest pressure, etc. The spot spray simulations can be based, in part, on the database of spot spray simulations. The database of spot spray simulations can also be used to form an agrochemical application plan.

An agrochemical application plan refers to a set of instructions or suggestions for maximizing crop yield or efficiency. The agrochemical application plan can provide a second agrochemical composition for application to the field, timing of applications, amount of application, etc. The agrochemical application plan can extend through a single planting, single planting season, or multiple seasons. For example, an agrochemical application plan can provide a rotation of herbicides to minimize the risk of resistant weeds. Such management strategies are preferably employed over a plurality of seasons. The agrochemical application plan can include a spot spray simulation as a component thereof.

Methods and systems of the disclosure include repeating steps, with or without variation. For example, in order to generate a database of spot spray simulations, it is possible to use methods and systems of the disclosure across multiple fields, multiple agrochemical composition, multiple application rates, etc.

One system of the disclosure includes at least one remote controlled or autonomous drone, at least one sensor attached to the drone, a plot of land configured to grow crops, and at least one agrochemical applicator configured to apply agrochemicals to the plot of land. As discussed, the agrochemical applicator can be a part of or separate from the drone. The system will additionally include a storage medium to record information from the at least one sensor as the drone scans the plot. Using the information from the at least one sensor to identify a presence of wanted or unwanted plant growth in the field, generate a spot spray simulation of wanted and unwanted plant growth, and provide an agrochemical application plan based on the spot spray simulation.

### Examples

Various field plots were prepared preferably having 20×50 ft size, or optionally 10×100 ft. Fields were treated with pre-emergent herbicides as described in Table 1:

| Treatment | Rate |
|---|---|
| Check | n/a |
| Bicep II Magnum ® | 2320 g ai/ha (1.5 qt/A) |
| Acuron ® | 1930 g ai/ha (2 qt/A) |
| Acuron ® | 2890 g ai/ha (3 qt/A) |

Drone Imagery was taken at V1, V3, V5 corn growth stages. Overall weed control was determined visually, and various models with different spray systems were assessed at various resolutions: 25×25 cm; 50×50 cm; and 100×100 cm.

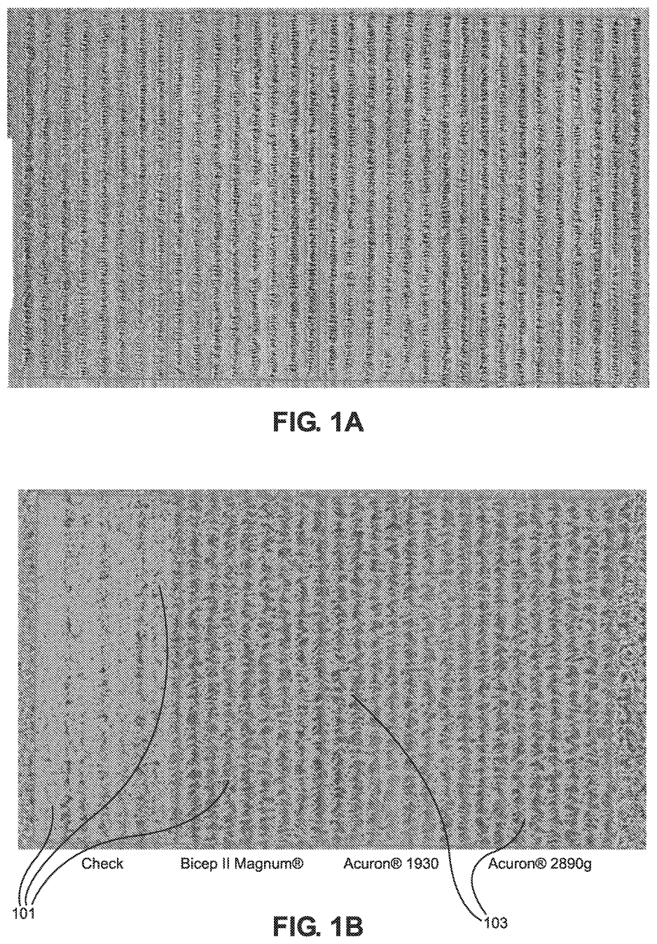

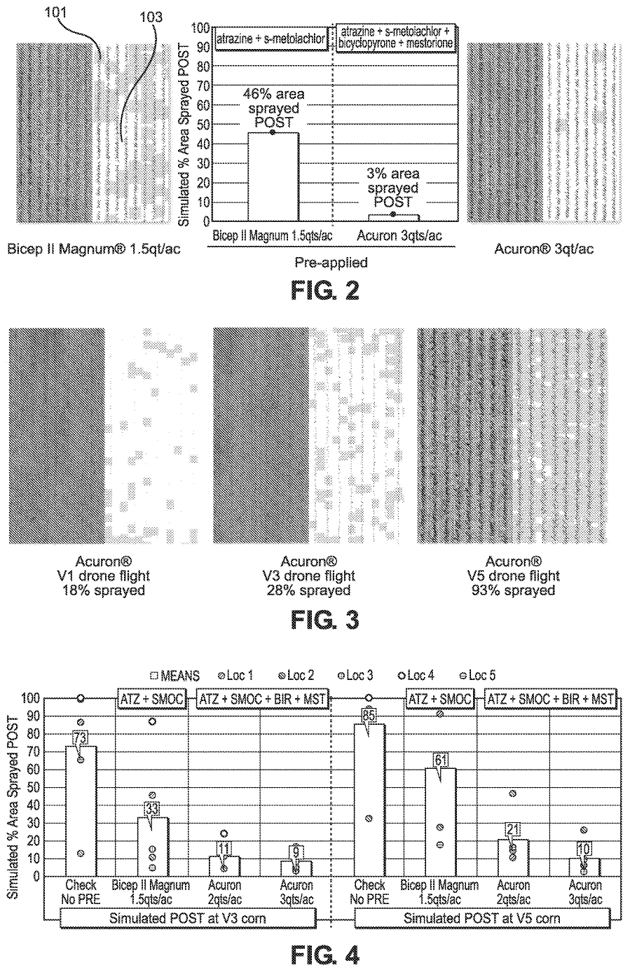

Stitched images of plots are provided in FIG. 1A. Identification of crops versus weeds using the images are provided in FIG. 1B. In FIG. 1B, green overlay indicates crop and orange overlay indicates weed. SAZs are indicated at 101 and NZs are indicated at 103. Using this system and above data, areas for postemergence optical spot spraying were identified, as shown in FIG. 2.

FIG. 3 illustrates weed growth and amount of spot spraying necessary to suppress weed growth. Specifically, at V1, 18% of the field requires spot spray; at V3 28% of the field requires spot spray; and at V5, 93% of the field requires spot spray.

The above system was implemented across five locations, and simulated post emergence treatment was calculated for V3 and V5 stages. The results are provided in FIG. 4.

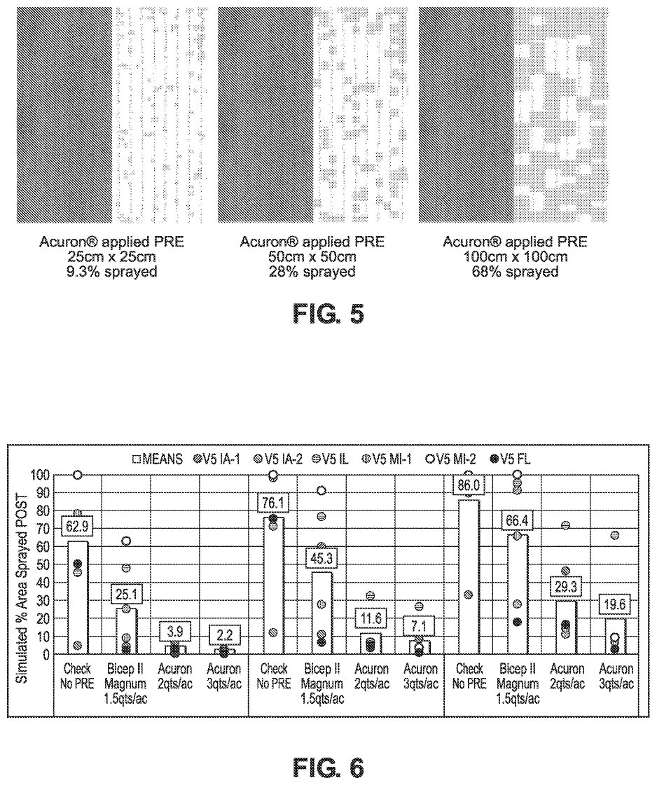

The simulations provided the flexibility to simulate various spray resolutions over the same field to determine area sprayed, as illustrated in FIG. 5. The impact of spray resolution on areas sprayed in simulated V5 application is provided in FIG. 6.

The provided system can model the impact of preemergence herbicides on the projected % area sprayed POST with an optical spot sprayer. Acuron® applied PRE provided better residual weed control, which results in a larger simulated POST herbicide savings at V3 (not shown) and V5 timings. Acuron® at V5 application provided 60 and 65% savings at 2 qt and 3 qt rate compared to check, respectively. Bicep II Magnum provided 31% savings.

In addition, the provided system modeled the impact various spray resolutions on the projected % area sprayed with an optical spot sprayer. Simulated % area sprayed varied by 20-40% depending on spray resolution. The use of Acuron® PRE consistently provided better weed control than no PRE or Bicep II Magnum, which reduces the impact of changing spray resolutions

It was determined that the use of drone imagery was an acceptable substitute for sprayer pass.

The above methodology was compared to a standard visual assessment utilizing 50×50 cm resolution at V5 application at three locations. The results are provided in Table 2:

| | MI-2 Location | | | IL Location | | | IA-2 Location | | |
|---|---|---|---|---|---|---|---|---|---|
| | Visual | Predicted % | Actual % | Visual | Predicted % | Actual % | Visual | Predicted % | Actual % |
| Treatment | Rating | Area | Area | Rating | Area | Area | Rating | Area | Area |
| Untreated | 0 | 100 | 100 | 0 | 100 | 71.165 | 0 | 100 | 11.47 |
| BIIM* | 55 | 45 | 90.96 | 4 | 96 | 76.565 | 0 | 100 | 10.72 |
| 1.5 qt | | | | | | | | | |
| Acuron ® | 98 | 2 | 6.5 | 94 | 6 | 32.545 | 25 | 75 | 17.85 |
| 2 qt | | | | | | | | | |
| Acuron ® | 99 | 1 | 3.21 | 98 | 2 | 26.5 | 95 | 5 | 8.44 |
| 3 qt | | | | | | | | | |

*BIIM = Bicep II Magnum ®

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

1. A method for evaluating the effectiveness of an agrochemical, the method comprising:
applying an agrochemical to a first field;
scanning the first field with a first drone having at least one sensor;
identifying a presence of wanted or unwanted plant growth in the first field; and
generating a first spot spray simulation for a second agrochemical based on the presence of wanted or unwanted plant growth in the first field, wherein the first spot spray simulation comprises:
a non-spot application zone (NZ), and
wherein when an unwanted plant has been identified, a simulated spot application zone (SAZ) is indicated over the position of the unwanted plant;
whereby the spot spray simulation can be used to evaluate the effectiveness of the agrochemical.

2. The method of claim 1, wherein the first drone is an aerial drone.

3. The method of claim 1, wherein the at least one sensor comprises a camera.

4. The method of claim 1, wherein the first spot spray simulation comprises a matrix and/or grid of wanted and unwanted plant growth

5. The method of claim 1, wherein the first agrochemical includes an herbicide and is applied pre-emergent.

6. The method of claim 1, wherein the first spot spray simulation includes determining an application amount for the second agrochemical based on the presence of wanted or unwanted plant growth in the field.

7. The method of claim 6, wherein the SAZ has a spray resolution value.

8. The method of claim 7, wherein the spray resolution value includes at least one of 25×25 cm; 50×50 cm; 100×100 cm, and a resolution in the range of 5-100 cm×5-100 cm.

9. The method of claim 1, wherein at least one or both of the agrochemical and the second agrochemical include an herbicide.

10. The method of claim 1, wherein the first field has been planted at a time T and wherein scanning occurs during at least one of 1-12 weeks after T, 2-4 weeks after T, 4-6 weeks after T, 6-8 weeks after T, or 9-10 weeks after T.

11. The method of claim 1, further comprising:
applying a third agrochemical to a second field, wherein the third agrochemical is different from the first agrochemical;
scanning the second field with a second drone having at least one sensor;
identifying a presence of wanted or unwanted plant growth in a portion of the second field; and
generating a second spot spray simulation for the second agrochemical based on the presence of wanted or unwanted plant growth in the field, whereby the second spot spray simulation can be compared to the first spot spray simulation to evaluate the effectiveness of the pre-emergent herbicides.

12. The method of claim 1, further comprising:
generating an estimate of an amount of the second agrochemical for application to the first field;
applying the second agrochemical by spot spraying to the first field;
measuring the amount of the second agrochemical applied; and
reporting a difference between the volume applied and the volume estimate.

13. A method, comprising:
(a) applying a first agrochemical composition to a field;
(b) scanning the field with a drone having at least one sensor;
(c) identifying a presence of wanted or unwanted plant growth in the field;
(d) generating and/or compiling a spot spray simulation, wherein the spot spray simulation comprises a matrix and/or grid of at least one non-spot application zones (NZ) and at least one simulated spot application zones (SAZ) comprising wanted and unwanted plant growth based on the identifying;
(e) creating a database of the spot spray simulation and the first agrochemical composition; and
(f) providing an agrochemical application plan based on the database.

14. The method of claim 13, wherein the agrochemical application plan provides a second agrochemical composition for application to the field.

15. The method of claim 13, wherein the applying the first agrochemical composition to a field is a pre-emergent treatment.

16. The method of claim 13, wherein (a)-(d) are repeated across multiple fields, multiple agrochemical compositions, and multiple application rates of the first agrochemical composition.

17. A system, comprising:
at least one remote controlled or autonomous drone;
at least one sensor attached to the drone;
a plot of land configured to grow crops;
at least one agrochemical applicator configured to apply agrochemicals to the plot of land;
a storage medium configured to store data captured by the at least one sensor; and
at least one processor in communication with the storage medium, and configured to:
(a) identify a presence of wanted or unwanted plant growth in the field,
(b) generate a spot spray simulation of wanted and unwanted plant growth based the identifying of the first processor, and
(c) provide an agrochemical application plan based on the spot spray simulation.
